(19) United States
(12) Patent Application Publication
(10) Pub. No.: US 2003/0211122 A1
(43) Pub. Date: Nov. 13, 2003

(54) Mucosal microparticle conjugate vaccine

(75) Inventors: Ingvar Sjoholm, Uppsala (SE); Lena Degling Wikingsson, Spanga (SE)

Correspondence Address: BURNS, DOANE, SWECKER & MATHIS, L.L.P., P.O. Box 1404, Alexandria, VA, 22313-1404, US

(73) Assignee: INNOVENTUS PROJECT AB

(21) Appl. No.: 10/459,525

(22) Filed: Jun. 12, 2003

Related U.S. Application Data
(62) Division of application No. 09/623,046, filed on Nov. 9, 2000.
Application No. 09/623,046 is a 371 of international application No. PCT/SE99/00277, filed on Feb. 26, 1999.

(30) Foreign Application Priority Data
Feb. 27, 1998 (SE) 9800615-8

(51) Int. Cl. A61K 39/112

(52) U.S. Cl. 424/258.1

(57) ABSTRACT

Mucosal, particularly oral, microparticle conjugate vaccines against certain pathogenic microorganisms, especially intracellular pathogenic microorganisms, are disclosed. An immunizing component of such a vaccine comprises protection-generating antigens derived from a certain pathogenic microorganism, such as Mycobacterium tuberculosis or Salmonella enteritidis, conjugated, possibly via a linker, to biodegradable microparticles, particularly starch microparticles, such as cross-linked starch microparticles, e.g. polyacryl starch microparticles. Further, a method of inducing protective immunity against a certain pathogenic microorganism in a mammal, and the use of protection-generating antigens derived from a certain pathogenic microorganism conjugated, possibly via a linker to biodegradable microparticles for the production of a mucosal microparticle conjugate vaccine are described.

The present invention relates to microparticle conjugate vaccines for mucosal, e.g. oral, administration to a mammal, including man. The vaccines are directed against a certain pathogenic microorganism, particularly an intracellular microorganism, such as Mycobacterium tuberculosis or Salmonella enteritidis. The invention also relates to a method of inducing protective immunity against such a microorganism, and to the use of protection-generating antigens derived from such a microorganism conjugated to biodegradable microparticles, for the production of the vaccines.

### BACKGROUND

Generally, vaccines today are formulated for parenteral administration. Only a few vaccines are used orally and then for specific purposes. Thus, oral cholera vaccines are intended to produce antibodies against the B-subunit CTB of the cholera toxin, causing diarrhea of the infected person, by disrupting the salt and water balance over the gut wall. The antibodies are supposed to inhibit the binding of the toxin via the CTB unit to a specific receptor (the GM1 receptor) in the epithelial wall. Moreover, some vaccines containing attenuated polio virus, with disputed efficacy, are approved to be used in some countries. However, no carrier system for oral use with isolated antigens has yet been approved for use in humans.

There are some obvious advantages with oral vaccines. They are easier to use than parenteral ones, as the administration does not require professional personnel, like nurses, and an oral administration avoids the stress caused by an injection, particularly in children. In addition, the manufacture of an oral product is easier and thereby cheaper than for a sterile, parenteral product. More important though, are the potentially improved effects of an oral vaccination over a parenteral one in newborns, where the immune system in the mucosal and gut regions develop earlier than in other parts of the body, where the parenteral vaccines are active. Also for elderly people the mucosal response is probably better after oral vaccination.

An important feature of an immune response is the memory function, which is mediated by specific B-cells, the differentiation and proliferation of which are induced by specific antigenic structures. A well functioning set of memory cells is needed to give the vaccinated person a life-long protection, experimentally identified by the so called booster effect obtained upon a late exposure to the antigen. Moreover, protection against an invading microorganism is also provided by a cellular response, which can be detected by the so called delayed-type hypersensitivity reaction, usually performed in the ears and footpads of mice. These immunological responses are frequently seen after parenteral vaccination. It has generally been assumed that oral vaccination gives a mucosal response, detected by the production of local antibodies of the subtype IgA (slgA). However, it would be desirable to obtain a mucosas, preferably oral, vaccine against pathogenic microorganisms which gives both a memory function and a cellular response in addition to a strong mucosal IgA production. Further, since cell-mediated immunity seems to be the most important defense against intracellular pathogens in a host, an efficient vaccine against such pathogens should stimulate the T-cell immune response.

Moreover, some experimental and epidemiological indications suggest that a cellular immune response predominately of the Th1-type is especially important to withstand viral and parasitic infections. A Th1 response is also thought to better mimic the response seen after a natural infection and to decrease the risks of later development of allergy.

A few vaccination studies have been performed with particulate antigens using the parenteral immunization route. Vordermeier et al. showed that a 38 kDa protein antigen from M. tuberculosis entrapped in the particulate adjuvant poly (DL-lactide co-glycolide) particles induced Th1-antigen specific humoral and cellular immune responses, which, however, did not protect against an intravenous challenge with M. tuberculosis (Vordermeier et al., 1995).

Earlier experimental vaccination studies with protective antigens derived from M. tuberculosis, i.e. secreted proteins, against tuberculosis have more or less successfully been carried out with different parenteral adjuvants e.g. Freund's incomplete adjuvants (FIA), dimethyldeoctadecylammonium chloride (DDA), poly (DL-lactide co-glycolide) particles, liposomes, aluminium hydroxide and RIBI adjuvants (Pal and Horwitz, 1992, Andersen, 1994a, Roberts et al. 1995, Vordermeier et al., 1995, Lindblad et al., 1997 and Sinha et al., 1997).

Until recently, alum precipitates, e.g. aluminum hydroxide, are the only adjuvants approved in the US and in Sweden for human use. In a recent study by Lindblad et al. (I 997), the use of aluminum hydroxide with secreted antigens from M. tuberculosis in an experimental vaccine was questioned. It induced a Th2 response, which, indeed, increased the susceptibility of the animals to a subsequent challenge with M. tuberculosis (Lindblad et al. 1997) This result shows that adjuvants available today for human use have to be replaced by new safe adjuvants for future acellular vaccines against intracellular pathogens, such as M. tuberculosis.

A new adjuvant was approved last fall consisting of synthetic, spherical virosomes with haemagglutinin and neuraminidase from influenza virus and inactivated hepatitis A-virus. The adjuvant is claimed to give less adverse reactions than the conventional aluminum adjuvants. (Glück R. 1995).

Biodegradable microparticles, particularly starch particles, such as cross-linked starch particles, have been disclosed in the prior art. The polyacryl starch microspheres conjugated to the protective antigens used in the experimental part of the present description of the invention, have previously been disclosed as parenteral adjuvants for antigen delivery (Degling and Stjärnkvist, 1995). The particles themselves do not induce an immune response, but are weak macrophage activators, (Artursson et al., 1985).

The lack of a general vaccination system for oral use is due to the problems associated with the administration of isolated antigens of protein or carbohydrate nature and the uptake of them through the gut epithelium and transport to the cells of the immune system. To start with, the antigens have to be protected against proteolytic degradation during the transport through the alimentary tract down to the immune competent regions in the gut. It is essential that the relevant epitopes of the antigens, at least, are preserved in order to be taken up, supposedly, by the M-cells in the Peyer's patches and subsequently transported to the antigen-presenting cells in the patches. Therefore, the vaccine has to be formulated in such a way that the antigen epitopes are protected until the antigens are taken up by the immune-competent cells.

### DESCRIPTION OF THE INVENTION

The present invention provides, unexpectedly, protection of antigens in the alimentary tract of mammals, as shown in mice, by conjugation of protection-generating antigens derived from pathogenic microorganisms to biodegradable microparticles, such as starch carriers, which are porous. The antigens obviously are not available inside the pores for the enzymes, neither are they able to diffuse out from the pores due to the covalent binding. It is, moreover, the current understanding that the M-cells and/or other endocytosing cells of the gut wall can take up and further transport only carriers of a narrow size in the submicro-meter region, or close to that, and with a specific surface structure. Unexpectedly, the mucosal microparticle conjugate vaccine of the invention seems to be partially degraded to such a size and structure, which is optimal in order to be taken up by the M-cells, and subsequently produce immune responses, which are protecting against a challenge of the relevant microorganism.

The invention, moreover, unexpectedly gives rise to such a cellular response—as detected by the delayed hypersensitivity test—and a mucosal sIgA response as well as a systemic IgM/IgG response, that give protection against the challenge of a microorganism, even when the improved stability of the antigens within the conjugated microparticulate vaccine is considered.

More precisely, the present invention is directed to a mucosal microparticle conjugate vaccine against a certain pathogenic microorganism, which comprises, as an immunizing component, a T-cell activating amount of protection-generating antigens derived from said microorganism conjugated, possibly via a linker, to biodegradable microparticles.

The biodegradable microparticles are preferably starch particles, such as cross-linked starch particles.

In a preferred embodiment of the invention the cross-linked starch particles are polyacryl starch microparticles.

In another preferred embodiment of the invention the mucosal vaccine is an oral vaccine.

The pathogenic microorganism is e.g an intracellular pathogenic microorganism, which in a preferred embodiment of the invention is selected from the group consisting of Mycobacterium tuberculosis and Salmonella enteritidis.

The certain intracellular pathogenic microorganism may be selected from a wide variety of different microorganisms such as Mycobacterium sp., Salmonella sp., Shigella sp., Leishmania sp., virus such as Rota virus, Herpes sp.. Vaccinia virus and influenza virus, Meningococces. Bordetella pertussis, Streptococcus sp,. enteroloxigenic Escherichla coli, Helicobacter pylori, Campylobacter jejuni, Toxoplasma gondii, Schistasoma sp., Listeria monocytogenes, Trypanosoma cruzi and other sp., Clamydia sp, HIV sp., etc.

The protection-generating antigens derived from a certain microorganism may be intracellular antigens, cell-wall antigens or secreted antigens.

Another aspect of the invention is directed to a method of inducing protective immunity against a certain pathogenic microorganism in a mammal, including man, comprising mucosal administration to said mammal of a T-cell, particularly of the Th1-type, activating amount of protection-generating antigens derived from said microorganism conjugated, possibly via a linker, to biodegradable microparticles, as an immunizing component.

In a preferred embodiment of the invention the mucosal administration is oral administration and the protection-generating antigens derived from said microorganism are secreted proteins from Mycobacterium tuberculosis or Salmonella enteritidis.

Yet another aspect of the invention is directed to the use of protection generating antigens derived from a certain pathogenic microorganism conjugated, possibly via a linker, to biodegradable microparticles for the production of a mucosal microparticle conjugate vaccine against said certain pathogen.

In a preferred embodiment of this aspect of the invention the mucosal vaccine is an oral vaccine, said antigens derive from Mycobacterium tuberculosis or Salmonella enteritidis, and the biodegradable microparticles are starch particles, such as cross-linked starch particles, including polyacryl starch microparticles.

In a most preferred embodiment of the invention the protection-generating antigens are secreted proteins from Mycobacterium tuberculosis (TB) Harlingen stran.

The T-cell activating amount of the conjugate of the invention depends on several factors such as physical, chemical and biological characteristics of the antigen, on the age and species of the individual mammal, and also the immunological and general physical status of the vaccinated individual. Recommended dosages will be given by the manufacturer based on clinical trials.

It should be understood that the conjugate of the invention may not only activate T-cells and particularly Th1 cells (even though the amount of the conjugate in a vaccine is calculated on the T-cell activation to ensure immunological memory), but may also give rise to a secretory IgA and a systemic IgM/IgG response.

The possible linker between the two components of the conjugate of the invention is used to facilitate the coupling reaction or to enhance the antigen presentation. The linker may be an amino-acid residue such as lysine, or an amino-acid sequence of a di-, tri-, or polypeptide.

The mucosal microparticle conjugate vaccine according to the invention may be presented in different pharmaceutical formulations depending on the actual intended route of administration, the specific conjugate and the solubility and stability of the antigen or antigens.

In order to guarantee the efficacy of the vaccine preparation it may be possible to do so by decreasing the degradation of the microparticle carrier by enzymes and/or acidic pH in the stomach and upper intestines, or by improving the uptake of the vaccine by the antigen-presenting cells, by modifying the formulation of the vaccine in different ways. Thus, e.g.

the cross-linking degree of the microparticles can easily be controlled by the derivatization degree of the starch used in the production of the microparticles, so that higher cross-linking will yield more resistant panicles, or

the size of the microparticles can be controlled during the production by the dispersion of the emulsion prior to the polymerization of the acrylic groups of the derivatized starch, so that larger particles will give a more stable product, or

the vaccine microparticles may be dispensed in hard gelatin capsules covered by gastro-resistant materials such as cellulose acetate phthalate, hydroxypropyl methylcellulose phthalate or acrylate polymers (Eudragit™), so that the vaccine is released after the transport through the stomach and the upper intestines, or

the vaccine microparticles may be individually covered by a gastro-resistant shell e.g. by coacervation-phase separation or multiorifice-centrifugal processes with e.g. shellac or cellulose acetate phthalate, so that the particles are protected during the transport through the stomach and upper intestines and thereafter released from the shells, or

the vaccine microparticles may be suspended in an alkaline buffer such as sodium bicarbonate, neutralizing the acidic pH in the stomach and the upper intestines, or

the vaccine microparticles may be compressed to a tablet with bulking agents such as lactose, disintegrants such as microcrystalline cellulose, lubricants such as magnesium stearate in such a way that the tablet is slowly disintegrated in the intestines making the vaccine microparticles available for uptake by the antigen-presenting cells, or

the vaccine microparticles may be compressed to a tablet with bulking agents such as lactose, disintegrants such as microcrystalline cellulose, lubricants such as magnesium stearate, which subsequently is covered by gastro-resistant materials such as cellulose acetate phthalate, hydroxypropyl methylcellulose phthalate or acrylate polymers (Eudragit™), or

the vaccine microparticle may be covered by a gel-forming material such as hydroxypropyl methylcellulose, which is protecting the vaccine through the transport through the stomach and upper intestines.

The present invention will be illustrated more in detail with the aid of the description of experiments and the results. However, the experiments should not be considered as limiting to the scope of the claimed invention.

### DESCRIPTION OF EXPERIMENTS

Experiment 1

Mice were immunized with polyacryl starch microspheres with covalently coupled extracellular proteins from Micobacterium tuberculosis (Harlingen strain) to investigate the potential of the conjugate as an oral vaccine. The humoral and cellular immune responses were investigated and the protection after challenge was determined.

Materials

The maltodextrin was a gift from Dr. Lars Svensson (Stadex, Malmö, Sweden), acrylic acid glycidyl ester was from Fluka (Buchs, Switzerland) and from Polysciences Inc. (PA., USA), N,N,N′,N′-tetramethylethylenediamine (TEMED) and 4-nitrophenylphosphate disodium salt were from Merck (Darnstadt, Germany), Biorad protein assay kit and horseradish peroxidase conjugated goat anti-mouse IgG were from Biorad (CA., USA), Freund's incomplete adjuvant was from Difco Laboratories (MI., USA). BCG vaccine was from Statens serm insitut (Copenhagen, Denmark), alkaline phospbatase conjugated human anti-mouse IgG/IgM was from Biosource (CA. USA), carbonyldiimidazole, bovine serum albumin (grade V), phenylmethyl sulphonyl fluoride, trypsin inhibitor, alkaline phosphatase conjugated goat anti-mouse IgA and 4-chloro-1-naphtol were from Sigma (St. Louis, Mo., U.S.A.).

Purification of Extracellular Proteins from M. tuberculosis

Mycobacterium tuberculosis (Harlingen strain) was grown for one, two and three weeks (corresponding to protein solution w1, w2 and w3) in Proskauer-Beck medium at Smittskyddsinstitutet in Stockholm. The three (w1,w2 and w3) protein solutions were treated separately during the purification process. The bacteria were removed by centrifugation at 5000 rpm for 30 minutes and the culture supernatant was filtered through two consecutive 0.2 μm filters and concentrated about 50-fold through a YM10 filter (Ainicon, Mass., U.S.A.). Ammonium sulphate (final concentration 4.24 M) was added to the concentrate during stirring. After centrifugation at 8000 rpm for 30 minutes the precipitate was dissolved in phosphate buffered saline (pH 7.0). The proteins (solution w1, w2 and w3) were dialyzed extensively in a Spectra/Por® dialysis membrane (Spectrum, Calif., U.S.A.) with a 3500 molecular weight cut off, against a buffer with 0.25 M boric acid and 0.15 M NaCl, pH 8.5. The protein concentration was determined with Coomassie Blue according to Bradford (Bradford, 1976). Bovine serum albumin was used as a standard.

The proteins were stored at −80° C. until further use.

Preparation of Polyacryl Starch Microparticles

The microparticles were prepared by polymerization of acryloylated starch in an emulsion, as previously described (Artursson et al., 1984 and Laakso et al., 1986). Briefly, 500 mg of acryloylated starch was dissolved in 5 ml of a 0.2 M sodium phosphate buffer, pH 7.5, 1 mM EDTA. Ammonium peroxidisulphate (200 μl) was added to give a final concentration of 0.8 M in the aqueous phase, which then was homogenized in 300 ml of toluene:chloroform (4:1). TEMED was used to initiate the polymerization. The microparticle composition is characterized by the D-T-C nomenclature (Hjecrtén 1962 and Edman et al., 1980)) and the amount of TEMED added. D represents acryloylated starch (g/100 mL); T is the total concentration of acrylic groups expressed as acrylamide equivalents (g/100 ml), and C is the relative amount of any additional cross-linking agent (eg., bis-acrylamide; % w/w). The microparticles used in this study had a D-T-C value of 10-0.5-0 and 100 μl of TEMED was added.

Coupling of Extracellular Proteins (TB) from Mycobacterium tuberculosis Microparticles

The extracellular proteins (TB) w1 and w2+w3 were coupled to microparticles using the CDI-method of Bethell et al. (Bethell et al., 1981) Microparticles (5 mg/ml) were activated with CDI (50 mg/ml) in dry DMF for 1 h at room temperature. After several centrifugal washings with DMF to remove unreacted CDI, particles (50 mg) were suspended in 10 ml of the coupling buffer, (0.250 M boric acid with 0.15 M NaCl, pH 8.5) containing mg amount of w1 or w2+w3. The mixture was rotated end over end at 4-6° C. for 48 h The TB-microparticles were then washed to PBS, filtered through a 10 μm filter and stored at 4-6° C. The amount of w1 and w2+w3 coupled was determined by amino acid analysis after acidic hydrolysis of the microparticles.

Particle Size Determination

The TB-particles were dried and photographed in a scanning electron microscope (S.E.M) (Jeol T330) at 5000 magnification. The particle size determined from scanning electron microscope photographs was ≦2 μm. In previous studies 98% of the particles had a diameter ≦2.5 μm determined with Coulter Counter (Degling and Stjämkvist, 1995).

Immunizations

Mice of the BALB/c ABom strain (Bomholigárd, Ry, Denmark), female, 8-10 weeks old, were used. Mice (5-6/group) were immunized orally by gastric intubation, four times on three consecutive days, with TB-microparticles containing w1 and w2+w3 proteins. Also groups of mice were immunized im with TB-microparticles containing w1 and w2+w3 proteins or with corresponding amount soluble w1 and w2+w3 in physiological saline, 0.1 ml. As one positive control, groups of mice were injected ip with w1+w2+w3 in Freund's incomplete adjuvant (FIA). As the other positive control mice were immunized sc with 0.1 ml diluted (with physiological saline) BCG vaccine When low doses of soluble w1+w2+w3 were administered, a carrier protein BSA 0.1% was co-administered to minimize adsorption of protein to the glassware.

For detailed information see Table 1-1, Immunization schedule.

Collection and Preparation of Blood Samples

Blood samples were collected on day 0, 7, 15, 34, 42, 49, 57 and 65 with heparinized capillary tubes from orbital plexus. The tubes were centrifuged and the sera collected and frozen at −20° C. until further use.

Collection and Extraction of Faeces

Faeces (4-6) from each mouse were collected at five consecutive days after immunization into Ellerman tubes and freeze dried. The dry weight was determined and a solution containing 50 mM EDTA, 5% dry milk, 2 mM phenylmethylsulfonyl fluoride and 0.1 mg soybean trypsin inhibitor/ml phosphate-buffered saline (PBS-A) was added (20 μl/mg faeces). Solid matter was mashed and separated by centrifugation at 13000 rpm for 15 minutes and the supernatants were frozen at −20° C. until further use.

Determination of anti-TB IgG and IgM and stgA with ELISA

A protein solution, an equal mixture of w1, w2 and w3 proteins, was diluted (18 μg/ml) with 0.05 M sodium bicarbonate buffer with 0.05% NaN₃ (pH 9.6) and Nune Immunoplate Maxisorb F96 plates were coated (100 μg/well) and incubated in a moist chamber at 4° C. over night. The plates were shaken dry and 1% OVA in 1 mM PBS-A, pH 7.4, was added (200 μl/well) and then incubated for 2 h in moist chamber at room temperature to avoid unspecific binding to the plates. After 5 washings with 0.05% Tween 20 in physiological saline with a Titertek microplate washer 120 (Flow Laboratories) the sera/faeces samples were added to the plates in series of twofold dilutions and incubated for 2 h and the plates were washed as before. An alkaline phosphatase-conjugated secondary antibody (human anti-mouse Ig G and Ig M or goat anti-mouse Ig A) diluted 1:1000/1:250 in PBS-A with 0.2% Tween 20 (PBS-T) was added (100 μl/well) and the plates were incubated for 2.5 h. After washings, the substrate, 4nitrophenylphosphate (1 mg/ml, in 10% diethanolamine buffer with 0.5 mM MgCl₂ and 0.02% NaN₃, pH 9.8 g) was added and the absorbance was measured after 10 minutes (12 min for Ig A) at 405 nm with a Multiscan MCC/340 microtiter plate spectrophotometer (Labsystem). Pooled negative serum was added to each plate (Ig G/Ig M measurements) as a negative control. An average of the absorbance values was calculated from the first well (1:20 dilution); mean=0.130, sd=0.045 n=19. A sample was considered to be positive if the value exceeded mean+3×sd, thus above 0.265. A positive sample (serum from mice immunized with 100 μg w1w2w3 in Freund's incomplete adjuvant) was also added to each plate, as a standard, and was treated in the same was as the other samples. Titers were given as −log₂ (dilution×10).

Delayed-Type Hypersensitivity (DTH) Test

In order to evaluate whether a cell mediated immune response against TB had developed, a DTH test was performed on day 52 ie. one week after the third immunization. The mice were given an intradermal injection (10 μl) in the left ear with the tuberculosis protein mixture w1-w3 (1 mg/ml) in physiological saline. As a control 10 μl physiological saline was injected in the right ear. The thickness of the ears was measured with a dial thickness gauge (Mitutoyo Scandinavia AB, Upplands Väsby, Sweden) before antigen challenge and 24, 48 and 72 h after. The DTH response was calculated according to (A₁−B₁/A₀)·100, where A₁=increase from time 0 of the ear thickness in the ear challenged with antigen at time t, B₁=increase from time 0 of the ear thickness in the ear challenged with physiological saline at time t and A₀=ear thickness in the ear challenged with antigen, before challenge (Degling and Stjämkvist, 1995).

Experimental Infection of Mice

Immunized mice and control mice were challenged at day 106 (18 days after the last immunization) with 5×10⁶ CFU M. tuberculosis (Harlingen strain) iv by the tail vein. The weight of the mice were determined before and 15 days after infection.

Determination of Protective Immunity

At day 121 (15 days after infection) infected mice were killed and the spleen and lung were removed aseptically. CPU of M. tuberculosis were determined by homogenizing each organ in PBS and serial 10 fold diluting the tissue homogenates before culturing the dilutions on duplicate plates of 7H10 agar. Colony forming units were counted after 3 weeks of incubation at 37° C.

SDS-PAGE and Immunoblotting

The proteins in fraction w1, w2, w3 and an equal mixture of w1w2w3 were separated on a PhastSystem® (Pharmacia, Uppsala, Sweden) gel electrophoresis apparatus using a 10 to 15% SDS PhastGel® (Pharmacia Biotech, Uppsala, Sweden). Gels were both silver stained and stained with Coomassie blue. The separated proteins were transferred onto a nitro-cellulose membrane (Pharmacia Biotech, Uppsala, Sweden) and incubated for 2 h in RT in a solution containing 5% dry milk in PBS-T on a shaker. After washings with PBS-T, 6 membranes were incubated for 20 h in RT on a shaker, in 0.5% OVA PBS-T with sera from group 1-6 (diluted 1:20). After washings with PBS-T the membranes were incubated for 2 h in 37° C. on a shaker, with the secondary antibody (horseradish peroxidase conjugated goat anti-mouse IgG, diluted 1:20 000 with 0.5% OVA in PBS-T). The substrate, 4-chloro-1-naphtol (10 mg dissolved in 3.3 ml MeOH and added to 16.7 ml 20 mM Tris, 500 mM NaCl buffer with 30 μl H₂O₃ (37%)), was added after washings with PBS-T. The reaction was stopped after 20 min with distilled water.

Statistics

Unpaired t-test was performed comparing means of two independent samples. A difference was considered significant if p<0.05.

RESULTS

Coupling of Tuberculosis Proteins to Polyacryl Starch Microparticles

From the first coupling of w1 protein fraction, 5.63 μg protein per mg microparticle was coupled (corresponding to a protein coupling yield of 23%) and from the subsequent coupling with the supernatant 1.38 μg w1 protein per mg microparticle (protein yield 6.4%) was coupled.

An additional coupling of protein fraction w1 was performed and 3.93 μg protein per mg microparticle was coupled (corresponding to a protein yield of 5.9%). From the coupling with fraction w2+w3, 4.16 μg protein per mg microparticle was coupled (corresponding to a protein coupling yield of 55%) and from the subsequent coupling with the supernatant 0.89 μg protein w2+w3 per microparticle was coupled (protein yield 10.1%).

Analysis of the Extracellular M. tuberculosis Proteins by SDS-PAGE and Immunoblotting

The three protein fractions i.e. w1, w2 and w3, were analyzed by SDS-PAGE in order to determine the size of the protein in the mixture used in the inmunization experiment. Several bands in the region 14.4-30 kDa and 43-94 kDa were observed (totally 12 bands) by SDS-PAGE analysis. There was no difference between the w1, w2 and w3 protein fractions. (Results not presented).

Delayed Type Hypersensitivity (DTH)

As seen in Table 1-2, there was an increase in the ear thickness in the group immunized orally with TB-microparticles after 24, 48 and 72 hours, however the increase was not significantly higher than in the other groups. The DTH-response induced in the group immunized im with TB-microparticles was, after 24 h, significantly higher than the control group. After 48 and 72 hours the DTH-response increased to be significantly stronger than both the DTH-response in the control group and in the group immunized im with free TB-antigen in physiological saline. After 72 hours the DTH-response in this group was also significantly higher than the response in the BCG group and comparative with the response in the group immunized with TB-antigen in Freund's incomplete adjuvant. Two mice in the control group showed a 40-50% increase in ear thickness and three mice did not respond at all. This explains the high mean and standard error (SD) within this group after 48-72 h.

The Humoral Immune Response

The group immunized with TB-microparticles im showed a response comparative with the group immunized with TB-proteins in Freund's incomplete adjuvant and the group immunized im with free TB-proteins in physiological saline. The response was also significantly higher than in the control and BCG groups. The group immunized orally with TB-microparticles did not give rise to a humoral IgG and IgM) response. (Table 1-3)

Mucosomal (sIg A) Immune Response

Preliminary results indicate a sIgA immune response 2 days after the third immunization in the groups given microparticles orally and im and in the group immunized with antigen in Freund's incomplete adjuvant. The response in the BCG group was lower. However further studies have to be performed to confirm these results.

Protection Experiments

The protection level was determined by two parameters, weight loss during infection and CFU of M. tuberculosis in the lung after infection. As seen in Table 1-4, both the mice in the control group and the vaccinated groups lose weight during infection. The CFU of M. tuberculosis in the lung after infection is presented in Table 1-5.

A protective immunity was manifested in animals immunized orally with TB-microparticles The reduction of viable M. tuberculosis in the lung was at least 10-100 fold as compared with the unimmunized control and comparable to the effect seen after immunization with BCG vaccine. (Table 1-5) The protection after intramuscular immunization with TB-microparticles was somewhat lower than the response after orally administered TB-microparticles although the reduction of viable M. tuberculosis in the lung was at least 10 fold. As seen in Table 1-5, no protective immunity was seen in animals immunized intramuscularly with free TB-antigen in physiological saline or intraperitoneally with TB-antigen together with FIA.

TABLE 1 1
Immunization Schedule.

| Immunization with: | Vaccine formula | Days of immunization |
|---|---|---|
| TB-microparticles oral | 1.5 mg microparticles with W1 + 1.5 mg microparticles with W2 + W3 | 0,1,2,22, 23,24 |
| | 8.45 μg protein W1(5.63 μg protein/mg microparticles) + 6.24 μg protein W2 + W3 | |
| | (4.16 μg protein/mg microparticles) | |
| | 1.5 mg microparticles with W1 + 1.5 mg microparticles with W2 + W3 | 43,44,45 |
| | 5.89 μg protein W1(3.93 μg protein/mg microparticles) + 6.24 μg protein W2 + W3 | |
| | (4.16 μg protein/mg microparticles) | |
| | 3 mg microparticles with W1 | 86,87,88 |
| | 57 μg protein W1 (19 μg protein/mg microparticles) | |
| TB-microparticles im | 0.5 mg microparticles with W1 + 0.5 mg microparticles with W2 + W3 | 0,22,43 |
| | 2.82 μg protein W1(5.63 μg protein/mg microparticles) + 2.08 μg protein W2 + W3 | |
| | (4.16 μg protein/mg microparticles) | |
| | 0.5 mg microparticles with W1 | 86 |
| | 8 μg protein W1 (19 μg protein/mg microparticles | |
| Free TB-antigen im | 2.82 μg protein W1 + 2.08 μg protein W2 + W3 | 0,22,43 |
| | 8 μg protein W1 | 86 |
| FIA with TB-antigen ip | 2.82 μg protein W1 + 2.08 μg protein W2 + W3 with Freund's incomplete | 0,22,43 |
| | adjuvant | |
| | 8 μg protein W1 with Freund's incomplete adjuvant | 86 |
| BCG vaccine sc | BCG vaccine (10⁷ bacteria) | 0,22,43,86 |
| Control/Unimmunized | — | — |

TABLE 1 2
Delayed Type Hypersensitivity Test

| | Increase in ear thickness | Increase in ear thickness | Increase in ear thickness |
|---|---|---|---|
| | 24 h after antigen challenge. | 48 h after antigen challenge. | 72 h after antigen challenge. |
| Immunization with: | (X ± SD) | (X ± SD) | (X ± SD) |
| TB-microparticles oral | 37.0 ± 27.9 | 45.3 ± 34.6 | 50.5 ± 32.1 |
| TB-microparticles im | 63.6 ± 35.3 | 93 ± 43.8 | 108.6 ± 47.3 |
| Free TB-antigen im | 28.3 ± 19.5 | 25.7 ± 9.4 | 31.7 ± 18.9 |
| FIA with TB-antigen ip | 55.2 ± 33.9 | 49.0 ± 21.6 | 57.0 ± 22.9 |
| BCG vaccine sc | 39.0 ± 14.9 | 56.5 ± 22.7 | 61.2 ± 14.7 |
| Control/unimmunized | 15.0 ± 21.8 | 10.2 ± 15.5 | 19.6 ± 27.0 |

TABLE 1 3
Antibody response (Antigen specific IgM and IgG)
Antibody titer presented as-log₂(dilution × 10)

| | Day 0 | Day 7 | Day 34 | Day 42 | Day 49 | Day 65 |
|---|---|---|---|---|---|---|
| Immunization with: | (X ± SD) | (X ± SD) | (X ± SD) | (X ± SD) | (X ± SD) | (X ± SD) |
| TB-microparticles oral | 0.7 ± 1.2 | 0.5 ± 1.2 | 0.7 ± 0.8 | 0 ± 0 | 0.7 ± 1.2 | 0 ± 0 |
| TB-microparticles im | 1.0 ± 1.7 | 0 ± 0 | 4.7 ± 0.8 | 3.2 ± 2.1 | 6.0 ± 1.3 | 7.3 ± 1.5 |
| Free TB-antigen im | 0.3 ± 0.6 | 0 ± 0 | 2.3 ± 1.5 | 1.5 ± 1.5 | 4.5 ± 2.1 | 5.2 ± 1.0 |
| FIA with TB-antigen ip | 0 ± 0 | 0.3 ± 0.8 | 3.2 ± 1.8 | 5.4 ± 1.1 | 10.6 ± 0.5 | 10.0 ± 1.4 |
| BCG vaccine sc | 0.3 ± 0.6 | 0 ± 0 | 4.0 ± 5.2 | 0 ± 0 | 0.2 ± 0.4 | 1.5 ± 2.1 |
| Control/unimmunized | 0 ± 0 | 0 ± 0 | 0.2 ± 0.4 | 0 ± 0 | 0 ± 0 | 1.0 ± 0 |

TABLE 1 4
Weight loss during infection with Mycobacterium tuberculosis
(Strain: Harlingen).
Challenge dose: 5 × 10⁶ bacteria

| | Weight loss in percent 15 days after |
|---|---|
| | (challenge) infection with M. tuberculosis. |
| Immunization with: | (x ± SD) |
| TB-microparticles oral | 13.0 ± 5.1 |
| TB-microparticles im | 25.6 ± 7.1 |
| Free TB-antigen im | 15.2 ± 1.2 |
| FIA with TB-antigen ip | 18.3 ± 4.6 |
| BCG vaccine sc | 9.6 ± 3.9 |
| Control/Unimmunized | 10.5 ± 2.1 |

TABLE 1 5
Protective immunity after infection with 5 × 10⁶ CFU M. tuberculosis (Harlingen strain).

| | M. tuberculosis CFU | M. tuberculosis CFU | M. tuberculosis CFU | M. tuberculosis CFU |
|---|---|---|---|---|
| | (counts) on culture | (counts) on culture | (counts) on culture | (counts) on culture |
| | plate. | plate. | plate. | plate. |
| | 10¹ dilution of organ | 10³ dilution of organ | 10⁴ dilution of organ | 10³ dilution of organ |
| | suspension before | suspension before | suspension before | suspension before |
| | culturing | culturing | culturing | culturing |
| Immunization with: | (x ± SD) | (x ± SD) | (x ± SD) | (x ± SD) |
| TB-microparticles oral | 1000 ± 0 | 46.5 ± 35.1 | 2.7 ± 2.6 | 0.1 ± 0.2 |
| TB-microparticles im | 1000 ± 0 | 45.6 ± 10.9 | 4.2 ± 3.9 | 0.7 ± 1.1 |
| Free TB-antigen im | >1000 | 716.5 ± 491.0 | 19.5 ± 7.0 | 3.5 ± 4.2 |
| FIA with TB-antigen ip | >1000 | 808.8 ± 427.5 | 12.1 ± 9.5 | 0.5 ± 0.5 |
| BCG vaccine sc | 762.4 ± 475.2 | 11.4 ± 5.8 | 0.9 ± 0.4 | 0 ± 0 |
| Control/unimmunized | >1000 | 818.7 ± 405.3 | 30.2 ± 12.7 | 3.7 ± 2.0 |

Experiment 2

Extracellular proteins were isolated from Salmonella enteritidis and covalently coupled to polyacryl starch microparticles. The immunogenicity of the conjugate after oral administration to mice and the induced protection against a challenge with live bacteria were followed.

Materials and Methods

Materials

In addition to those items specified in Experiment 1, Bacto-tryptone and Bacto-yeast-extract were from Difco (MI., USA.), alkaline phosphatase-conjugated goat anti-mouse IgA and mouse IgA-kappa from Sigma (MO., USA.) and RPMI 1640, HEPES and glutamine were from Life Technologics LTD (Paisley, Scotland).

Purification of Extracellular Protein from Salmonella enteritidis

Salmonella enteritidis wild-type was inoculated in 2 ml Luria-Bertani (LB) broth (1% Bacto-tryptone/0.5% Bacto-yeast-extract/1% sodium chloride) and grown with shaking, 200 rpm, at 37° C. overnight. The next day the culture was diluted in 500 ml LB and grown under the same conditions until OD=1. After centrifugation (1,500×g for 60 min at 4° C.) the bacterial pellet was resuspended in RPMI 1640 with 20 nM HEPES and 4 mM glutamine. The mixture was shaken (200 rpm) at 37° C. for 2 h and thereafter the bacteria were removed by centrifugation at 1,500×g for 1 h at 4° C. The culture supernatant was filtered through a 0.22 μm Millipore express filter and concentrated and transferred into coupling buffer (0.250 M boric acid with 0.15 M NaCl, pH 8.5) by filtering through a YM 10 000 cut off Stirred Cell Ultrafilter, Amicon (MA., USA.). The protein concentration was determined with Coomassie Blue according to Bradford (Bradford, 1976 ) and with a ready prepared reagent from Bio-rad, using bovine serum albumin as a standard.

Preparation of Polyacryl Starch Microparticles, Conjugation of Salmonella Antigen and Characterization of the Antigen-Particle Conjugate

The Salmonella antigen-containing microparticles of polyacryl starch were prepared and characterized as described in Experiment 1.

Immunization Procedures

The mice, from own breeding of the Balb/c strain, were divided into 5 groups (4 mice/group)

(In the challenge experiment, 6-12 mice were included in each group.) In the first group each mouse was immunized ip with 10.5 μg protein in 0.1 ml Freund's adjuvant. The second group received an im injection with 10.5 μg protein conjugated to 1 mg microparticles. Mice in the third group were immunized orally by gastric intubation, with 31.5 μg protein conjugated to 3 mg microparticles divided in doses given on 3 consecutive days. Group four was an untreated control group and group five was a hyperimmunization group, which received 50 μg protein in 0.1 ml Freund's adjuvant (30 μg proteins as booster dose). Boosters were given after 21 days.

Collection and Handling of Blood and Faeces Samples

The sampling procedures used for the collection and handling of blood and faeces are presented in Experiment 1.

Assessment of Immune Responses

The analyses of the systemic IgG response as well as the mucosal IgA response were performed by conventional ELISA techniques, which are described in Experiment 1. The cellular response was analyzed by the delayed-type hypersensitivity test (DTH-test) as presented in Experiment 1.

Challenge of Immunized Mice

Challenge with Salmonella enteritidis (3×10⁴ CFU/mouse) was performed 6 weeks after booster, Mice were killed 7 days after challenge. Liver and spleen homogenates were incubated on LB-agar plates overnight and the number of CFU was counted.

Results

Characterization of the Antigen-Microparticle Conjugate

The conjugated starch microparticles contained 10 mg Salmonella antigen per mg. All particle preparations used contained more than 90% particles with a diameter less than 3.3 mm.

Humoral Immune Responses

The IgG/IgM response in serum in the group immunized orally with Salmonella proteins coupled to polyacryl starch microparticles was comparable with the response induced in the group immunized with proteins in Freund's adjuvant, but lower than the response induced when particles were administered im (Table 2-1) Similarly, the specific IgA response in faeces was comparable in the group immunized orally with Salmonella proteins coupled to polyacryl starch microparticles and the group immunized with proteins in Freund's adjuvant, showing a peak at day 27 and 28, whereas the specific IgA response induced in the group immunized im was lower (Table 2-2).

Cellular Immune Response

A relatively high, continuous increase in the ear thickness was detected in the group immunized orally with microparticles. The response was lower than that obtained with the positive control (in Freund's adjuvant) and comparable to the response induced in the group immunized im (Table 2-3).

Challenge of Immunized Mice

The results from the challenge of the immunized mice with live Salmonella bacteria are shown in Tables 2-4 and 2-5. A reduction in CFU was seen in the groups immunized orally with antigen-coupled microparticles, microparticles with soluble antigen or with soluble antigen alone, compared to the control group. The best protection was seen in the groups immunized with antigens together with or conjugated to starch microparticles. This was also seen when studying the average weight loss, which showed a 10.3% decrease for the control group, 4.0% decrease for the group immunized orally with soluble antigen, 3.6% decrease for the group immunized orally with microparticles with soluble antigen and 1.8% decrease for the group immunized orally with antigen-coupled microparticles (not presented in any table).

The results of this study show that secreted antigens derived from Salmonella conjugated to polyacryl microparticles may be administered as an oral vaccine capable of inducing both local secretory and systemic immune responses. Moreover, the a strong specific IgA response was observed in this study, although with significant interindividual variations.. The good protection against a challenge was also indirectly shown by following the weight loss after the challenge. The group treated orally with the antigen-conjugated microparticles lost significantly less in weight (1.8%) compared to the control group, not treated at all, loosing 10.3% in weight after the challenge.

TABLE 2 1
Specific humoral response in serum after immunization
with S. enteritidis antigens in
different formulations.
Titers are given as mean +/− S.E.M. (n = 4).

| Way of administration | Titer | |
|---|---|---|
| Antigen formulation | Day 0 | Day 35 |
| Oral immunization | 0.0 | 5.5 +/− 0.3 |
| Antigens conjugated in | | |
| microparticles | | |
| Im immunization | 0.0 | 9.0 +/− 0.4 |
| Antigens conjugated in | | |
| microparticles | | |
| Ip immunization | 0.0 | 7.0 +/− 0.0 |
| Soluble antigens in | | |
| Freund's adjuvant | | |

TABLE 2 2
Specific mucosal response (IgA) in faeces after immunization
with S. enteritidis antigens
in different formulations.
Values are given as means +/− S.E.M. (n = 4).

| Way of administration | IgA (ng/mg faeces) | | |
|---|---|---|---|
| Antigen formulation | Day 26 | Day 27 | Day 28 |
| Oral immunization | 1.1 +/− 0.5 | 2.1 +/− 0.55 | 2.25 +/− 0.8 |
| Antigens conjugated in | | | |
| microparticles | | | |
| Im immunization | 0.4 +/− 0.2 | 0.9 +/− 0.35 | 0.6 +/− 0.2 |
| Antigens conjugated in | | | |
| microparticles | | | |
| Ip immunization | 1.3 +/− 0.55 | 1.4 +/− 0.5 | 2.4 +/− 0.75 |
| Soluble antigens in | | | |
| Freund's adjuvant | | | |

TABLE 2 3
Cellular immune response (as test on delayed type hypersensitivity)
after immunization with S. enteritidis antigens in different formulation.
Results are given as mean % increase in thickness of challenged
ears, +/− S.E.M. (n = 2 − 4)

| Way of administration | % increase, hours after challenge | | |
|---|---|---|---|
| Antigen formulation | 24 | 48 | 72 |
| Oral immunization | 41 +/− 10 | 67 +/− 3 | 99 +/− 15 |
| Antigens conjugated in | | | |
| microparticles | | | |
| Im immunization | 107 +/− 13 | 138 +/− 24 | 179 +/− 12 |
| Antigens conjugated in | | | |
| microparticles | | | |
| Ip immunization | 190 +/− 22 | 209 +/− 25 | 214 +/− 21 |
| Soluble antigens in | | | |
| Freund's adjuvant | | | |
| Non-immunized | 10 +/− 1 | 23 +/− 4 | 38 +/− 2 |
| mice (controls) | | | |

TABLE 2 4
Colony forming units (CFU) in liver of mice immunized
with S. enterititidis antigens
after challenge with 3 × 10⁴ CFU.
The mice were challenged 6 weeks after booster and
killed 7 days after challenge. The livers
were homogenized and total CFU counted after incubation
over night in LB-agar. The results
are presented as geometric mean and range; n is given in parenthesis.

| Way of administration | CFU in liver | |
|---|---|---|
| Antigen formulation | Mean | Range |
| Oral administration | 1.8 × 10³ (6) | 2-3.12 × 10⁶ |
| Antigens conjugated in | | |
| microparticles | | |
| Oral administration | 0.69 × 10³ (6) | 1-1.5 × 10⁴ |
| Soluble antigens with | | |
| microparticles | | |
| Oral administration | 4.5 × 10³ (6) | 1-5.0 × 10⁴ |
| of soluble antigens | | |
| Non-immunized mice | 2.6 × 10⁶ (12) | 1.1 × 10⁵-1.9 × 10⁷ |
| (Controls) | | |

TABLE 2 5
Colony forming units (CFU) in spleen of mice
immunized with antigens from S.
enterititidis after challenge with 3 × 10⁴ CFU.
The mice were challenged 6 weeks after booster and
killed 7 days after challenge. The livers
were homogenized and total CFU counted after incubation
over night in LB-agar. The results
are presented as geometric mean and range; n is given in parenthesis.

| Way of administration | CFU in spleen | |
|---|---|---|
| Antigen formulation | Mean | Range |
| Oral administration | 3.22 × 10³ (6) | 3-2.48 × 10⁶ |
| Antigens conjugated in | | |
| microparticles | | |
| Oral administration | 1.43 × 10³ (6) | 1-5.70 × 10⁴ |
| Soluble antigens with | | |
| microparticles | | |
| Oral administration | 6.04 × 10³ (6) | 1-3.30 × 10⁵ |
| of soluble antigens | | |
| Non-immunized mice | 2.32 × 10⁶ (12) | 2.3 × 10⁵-1.5 × 10⁷ |

### References

Andersen, P., Effective vaccination of mice against Mycobacterium tuberculosis infection with a soluble mixture of secreted mycobacterial proteins. Infec. Immun., 62 (1994a) 2536-2544.

Artursson, P., Edman, P., Laakso, T. and Sjöholm I., Characterization of polyacryl starch microparticles as carrier for proteins and drugs. J. Pharm. Sci., 73 (1984) 1507-1513.

Artursson. P., Edman, P. and Sjdhölm, I., Biodegradable microspheres II: immune response to a heterologous and an autologous protein entrapped in polyacryl starch microparticles. J. Pharmacol. Exp. Ther, 234 (1985) 255-259.

Bethell, G. S., Ayers, J. S., Hearn, M. T. W. and Hancock, W. S. Investigation of the activation of various insoluble polysaccarides with 1,1′-carbonyldiimidazole and of the properties of the activated matrices. J. Chromatography. 219 (1981) 361-372.

Bradford, M. M., A rapid and sensitive method for the quantitation of microgram quantities of protein utilizing the principle of protein-dye binding Anal. Biochem., 72 (1976) 248-254.

Degling, L. and Stjämkvist, P., Biodegradable microspheres XVIII: the adjuvant effect of polyacryl starch microparticles with conjugated human serum albumin. Vaccine., 13 (1995) 629-636.

Edman P., Ekman, B. and Sjdhölm, I. Immobilization of proteins in microspheres of biodegradable polyacryidextran. J. Pharm. Sci. 69 (1980) 838-842.

Glück, R., Liposomal presentation of antigen for human use. In Vaccine Design: The subunit and adjuvant approach. Edited by Michael F. Powell and Mark J. Newman. Plenumn Press New York 1995. pp 325-345.

Hjenén, S. Molecular sieve chromatography on polyacrylamide gels prepared according to a simplified method. Arch. Biochem. Biophys. suppl. (1962) 147-151.

Laakso, T., Atursson, P. and Sjöholm, I., Biodegradable Microspheres IV; Factors affecting the distribution and degradation of polyacryl starch microparticles. J. Pharm. Sci., 75 (1986) 962-967.

Lindblad, E. B.. Elhay, M. J., Silva. R. Appelberg, R. and Andersen, P. Adjuvant modulation of immune responses to tuberculosis subunit vaccines. Infection Immnunity, 65,(1997) 623-629.

Pal P. G. and Horwitz, M. A., Immunization with extracellular proteins of Mycobacterium tuberculosis induces cell-mediated immune responses and substantial protective immunity in a guinea pig model of pulmonary tuberculosis. Infect Immun., 60 (1992) 4781-4792.

Roberts, A. D., Sonnenberg. M. G., Ordway, D. J., Furney, S. K., Brennan, P. J., Belisle, J. T. and Orme I. M. Characteristics of protective immunity engendered by vaccination of mice with purified culture filtrate protein antigens of Mycobacterium tuberculosis. Immunology 85 (1995) 502-508.

Sinha, R. K., Verma, I. and Khuller, G. K. Immunobiological properties of a 30 kDa secretory protein of Mycobacterium tuberculosis H37 Ra. Vaccine 15 (1997) 689-699.

Vordermeier, H. M., Coombes A. G. A., Jenkins, P, McGee, J. P., O'Hagan, D. T., Davis, S. S. and Singh, M. Synthetic delivery system for tuberculosis vaccines: immunological evaluation of the M. tuberculosis 38 kDa protein entrapped in biodegradable PLG microspheres. Vaccine 13 (1995) 1576-1582.

1. Mucosal microparticle conjugate vaccine against a certain pathogenic microorganism, which comprises, as an immunizing component, a T-cell activating amount of protection-generating antigens derived from said microorganism conjugated, possibly via a linker, to biodegradable microparticles.

2. Vaccine according to claim 1, wherein the biodegradable microparticles are starch particles, including cross-linked starch particles.

3. Vaccine according to claim 2, wherein the cross-linked starch particles are polyacryl starch microparticles.

4. Vaccine according to any one of claims 1-3, wherein the mucosal vaccine is an oral vaccine.

5. Vaccine according to any one of claims 1-4, wherein the pathogenic microorganism is an intracellular pathogenic microorganism.

6. Vaccine according to claim 5, wherein said intracellular pathogenic microorganism is selected from the group consisting of Mycobacterium tuberculosis and Salmonella enteritidis.

7. Method of inducing protective immunity against a certain pathogenic microorganism in a mammal, including man, comprising mucosal administration to said mammal of a T-cell activating amount of protection-generating antigens derived from said microorganism conjugated, possibly via a linker, to biodegradable microparticles, as an immunizing component.

8. Method according to claim 7, wherein the mucosal administration is oral administration and the protection-generating antigens derived from said microorganism are secreted proteins from Mycobacterium tuberculosis or Salmonella enteritidis

9. Use of protection-generating antigens derived from a certain pathogenic microorganism conjugated, possibly via a linker, to biodegradable microparticles for the production of a mucosal microparticle conjugate vaccine against said certain pathogen.

10. Use according to claim 7, wherein the mucosal vaccine is an oral vaccine, said antigens derive from Mycobacterium tuberculosis or Salmonella enteritidis, and the biodegradable microparticles are starch particles, including cross-linked starch particles and polyacryl starch microparticles.
